(19) United States
(12) Patent Application Publication
(10) Pub. No.: US 2002/0192289 A1
(43) Pub. Date: Dec. 19, 2002

(54) Polymer gel for cancer treatment

(76) Inventors: Ji Zheng, Houston, TX (US); Feng Chu, Houston, TX (US)

Correspondence Address: Ji Zheng, 5606 Bissonnet, #18, Houston, TX, 77081, US

(21) Appl. No.: 10/173,354

(22) Filed: Jun. 15, 2002

Related U.S. Application Data
(60) Provisional application No. 60/298,943, filed on Jun. 18, 2001.

(51) Int. Cl. A61N 1/30; A61K 31/74

(52) U.S. Cl. 424/486; 604/20

(57) ABSTRACT

A method is disclosed for cancer treatment based on using a solid polymer gel to completely block blood vessels of tumor. A polymer aqueous solution is injected into blood vessels and formed a solid gel in blood vessels of tumor by applying electromagnetic radiation or temperature source at tumor tissue to inducing crosslinking or phase transition. The tumor cells starve and perish because of without nutrients and oxygen provided by vascularization and metastasis can also be prevented because polymer gels blocks tumor cells to shed into blood circulation, when the blood vessels of tumor are completely blocked by the solid polymer gels. Also, anti-cancer drug including chemotherapy drug, radiation drug or anti-angiogenic drug can be mixed or conjugated with the polymer in polymer aqueous solution to be locally delivered to the tumor after polymer gel formation in the blood vessels of tumor of human or animal.

### BACKGROUND OF THE INVENTION

1. Field of the Invention

The invention generally relates to the fields of cancer biology, cell biology and polymer chemistry. The invention specifically relates to the field of cancer treatment. The invention provides a method, composition and process for cancer treatment and preventing metastasis of a carcinoma.

### RELATED ART

The present invention is related to a method of cancer therapy by blocking or coating blood vessels of tumor with a solid polymer gel. A polymer aqueous solution is injected into tumor blood vessels and exposed to a active species which is applied to tumor site and produce free radicals to initiate crosslinking and gel formation of polymer , and thus induce tumor cells starving and perishing without nutrients and oxygen provided by vascularization. However, the polymer aqueous solution in the normal blood vessels without active species initiation will keep liquid state and be degraded by hydrolysis or enzyme. The present invention is also related to a method to completely block metastasis (second tumor, tumor transformation, tumor cell shed into circulation) by the solid polymer gel formed in the tumor blood vessel as a barrier to cut off blood circulation through tumor site.

The capacity to induce the growth of new capillaries in the host is a characteristic common to most solid malignant tumors. This phenomenon has been called “tumor angiogenesis”. Tumor cell survival, growth and metastasis require persistent new blood vessel growth (angiogensis)(Risau, W., 1997, Zetter, B. R., 1998, Bicknell, R., 1997). The growth of a solid tumor is largely dependent on oxygen and nutrients provided by the vascularization. Folkman hypothesized (Folkman, J., 1974 & Folkman, J. et al, 1973) that the limitations imposed by diffusion of oxygen and nutrients prevent a tumor nodule from growing beyond a few millimeters in diameter unless the tumor nodule is penetrated by new capillaries. The hypothesis implies that inhibition of angiogenesis might control tumor growth and a decrease in angiogenesis in a given metastatic tumor should produce a decrease in the number of tumor cells shed into the circulation and a corresponding decrease in the number of metastatic colonies that arise downstream. For this reason, “anti-angiogenesis” was proposed as a potential therapeutic approach (Folkman, J., 1972).

A strategy has emerged to treat cancer by inhibiting angiogenesis (Folkman, J., 1997). It is of interest that some agents-including taxol, tamoxifen, and adriamycin, which are already in clinical use as anti-tumor agents are being found to have anti-angiogenic activity(Bolotti, D., et al, 1996, Gagliardi, A. R., et al, 1996, Steiner, R., 1992). Teicher and colleagues have reported that the combination of conventional chemotherapeutic agents with anti-angiogenic agents gave better results in reducing tumor metastases that was found with either agent alone (Teicher, B. A., et al., 1992, Teicher, B. A., 1994). Although clinical trials with a few anti-angiogenic agents such as the fungal drug analog TNP-470 (Figg, W. D., 1997) and the calcium signaling inhibitor carboxyamidotriazole (Kohn, E. C., 1996) are currently in progress, few have been completed. Several anti-angiogenic agents such angiostatin and endostatin have not yet entered clinical trials. Tumor-derived angiogenesis inhibitors, angiostatin (O'Reilly, M. S., 1994) and endostatin (O'Reilly, M. S., 1997) which isolated from tumor, inhibits primary tumor growth as well as establishment and growth of metastases. Controlled release polymers which are capable of releasing large molecules such as angiogenic inhibitors were also developed (Langer, R., et al., 1976). A considerable amount of research (Zetter, B. R., 1998) has been also conducted on the anti-angiogenic properties of naturally occurring angiogenic inhibitors, such as, thrombospondin, interferon and metalloproteinase inhibitors. It is shown that angiogenesis and concomitant tumor growth can also be inhibited by anti-adhesive peptides antibodies (Brooks, P. C., 1994) as well as by peptide antagonists that block the interaction of these integrins with their extracellular matrix ligand (Brooks, P. C., Montgomery, A. M., et al, 1994). An application of this technology has been reported in a recent publication in which cyclized RGD-containing peptides that inhibit integrin function were displayed on bacteriophage (Pasqualini, R., et al., 1997). Peptides have been described that selectively target angiogenic endothelial cells (Arap, w. et al., 1998). Conjugates made from these peptides and the anti-cancer drug doxorubicin induces tumor regression in mice with a better efficiency and a low toxicity than using doxorubincin alone.

However, the most commonly held theories of angiogenesis predict that, in most cases, some tumor may remain after a long-term of anti-angiogenic treatment, because the tumor that reaches a small-enough size (about 2 mm thick) is no longer dependent on angiogenesis. If the anti-angiogenic treatment were discontinued, the tumor would be expected to regrow. The arrival of new blood vessels allowed continued three-dimensional expansion of tumor.

Although these strategies described above are being used to try to reduce tumor angiogenesis with angiogenic inhibitors, peptide and combination of inhibitor with chemotherapy drug, none of them has been able to completely and rapidly obstruct and eliminate the tumor blood vessels to let tumor cells starve and perish in a short-term treatment. Moreover, these approaches may suffer from the problems of their nonselective toxic effects on normal tissue and secondarily acquired drug resistance that bedevils conventional cancer therapies.

The present invention fulfills the need for a simple, effective, non or low-toxic and useful method for cancer therapy using solid polymer gel formed in the tumor blood vessel to completely block blood flow in tumor site to induce tumor cells starving and perishing and further prevent tumor cells from migration into blood circulation for completely cancer treatment because the polymer gel in the tumor blood vessel forms a barrier to cut off the blood circulation through tumor tissue. The polymer gel is formed in the blood vessels of tumor by injecting polymer aqueous solution (can be polymer or oligomer or monomer molecule) into blood vessels and then exposing to active species (temperature, light, etc) applied at tumor site.

### BRIEF SUMMARY OF THE INVNTION

The present invention provides a method of treating a solid tumor by blocking or coating blood vessels of tumor with a solid polymer gel which is formed from a polymer aqueous solution by exposuring to a active species applied in tumor site. The polymer aqueous solution is able to form a solid gel in blood vessels of tumor upon the inter- or intra-molecule crosslinking or phase transition.

An embodiment of the present invention is a method of treating a solid tumor comprising the steps of: providing a polymer aqueous solution comprising one or more crosslinkable oligomers or polymers, initiator and crosslinking agents; injecting the said polymer aqueous solution into blood vessels of cancer patient or animal at tumor site; and applying electromagnetic radiation to tumor tissue site and generating free radicals to initiate polymer crosslinking and whereby a solid gel formation to block or coat the blood vessels of tumor.

Another embodiment of the invention, the electromagnetic radiation source include, but not limited to x-rays, ultrasound, infrared radiation, far infrared radiation, ultraviolet radiation, long-wavelength ultraviolet radiation, visible light, laser beam and y-ray radiation.

Still another embodiment of the present invention, the polymer aqueous solution further comprises a photoinitiator, wherein the photoinitiator may be erythrosine, phloxime, rose Bengal, thonine, camphorquinone, ethyl eosin, eosin, methylene blue, riboflavin, 2,2-methyl-2-phenylacetophenone, 2-methoxy-2-phenylacetophenone, 2,2dimethoxy-2-phenylacetophenone, and other acetophenone derivatives.

Yet another embodiment of the present invention, the x-rays, ultrasound, infrared radiation, far infrared radiation, ultraviolet radiation, long-wavelength ultraviolet radiation, visible light, laser beam or γ-ray radiation is externally or directly applied to the tumor site.

Further another embodiment of the present invention, the polymer aqueous solution comprises a cocatalyst, wherein cocatalyst may be N-methyldiethanolamine, N, N-dimethyl benzylamine, triethanolamine, triethylamine, dibenzylamine, N-benzylethanolamine, and N-isopropyl benzylamine.

An embodiment of the present invention is a composition comprising a crosslinkable polymer and photoinitiator and cocatalyst. In further embodiments, the crosslinkable polymer may be any kind of synthetic or nature polymers with or without polymerizable groups.

An embodiment of the present invention is a method of treating tumor comprising the steps of: providing a polymer aqueous solution comprising one or more polymerizable polymer; injecting the said polymer aqueous solution into tumor blood vessels of cancer patient or animal at tumor site; and applying temperature chang to tumor site by heater or cooler to cause polymer solidification and gelation by phase transition of polymer solution, then the solid polymer gel block or coat the blood vessels of tumor to induce tumor perish because of defect of nutrient. In further embodiment of the present invention, the polymer may be synthetic or nature polymers or macromolecules, wherein the polymer is, but not limited to poly (propylene fumarate)/poly(ethylene glycol)-dimethacrylate/β-tricalcium phosphate, acrylamide, N-isopropylacrylamide, gelatin, collagen, chitosan and polysaccharide.

Another embodiment of the present invention, the temperature source is liquid gas (for example, liquid nitrogen etc), solid gas (for example, dry ice, etc), ultrasound, electronic field and other heater or cooler which can heat or cool tissue temperature to above or below 37° C.

An embodiment of the present invention is a method of treating tumor comprising the steps of: providing a mixture solution comprising gel formable oligomer or polymer aqueous solution and anti-cancer drug; injecting the said mixture solution into blood vessels of cancer patient or animal at tumor site; and exposing the mixed solution to electromagnetic radiation or temperature change and whereby cause solid gel formation in blood vessels of tumor to encaposulate anti-cancer drug which will locally release to tumor site.

### BRIEF SUMMARY OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1. The crosslinking and solidification of polymer aqueous solution to form solid polymer gel upon exposure to radiation or temperature change.

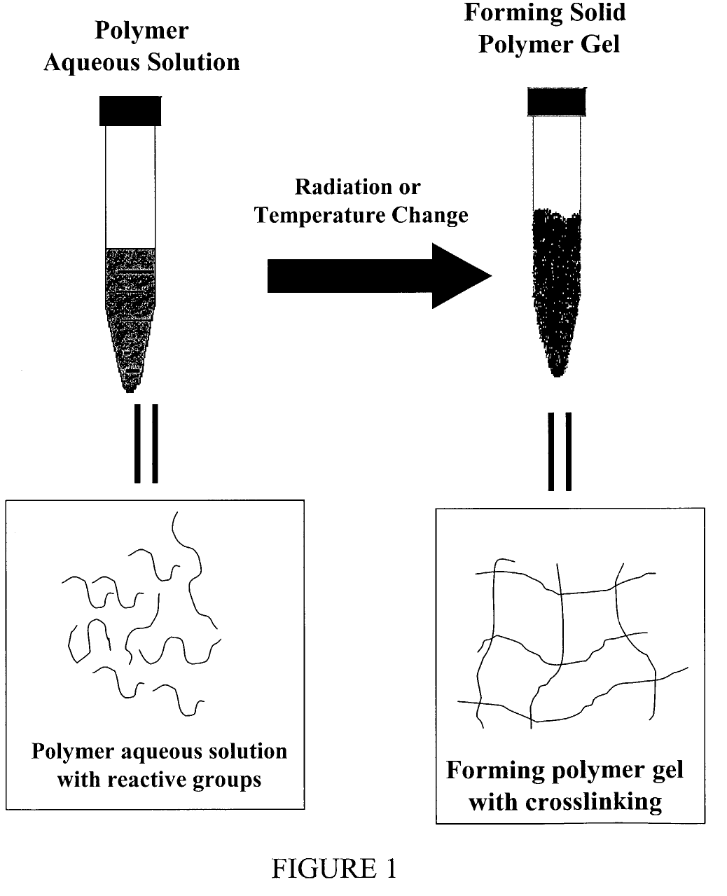

FIG. 2. The working model of completely blocking blood vessels at tumor site by the polymer gel formation.

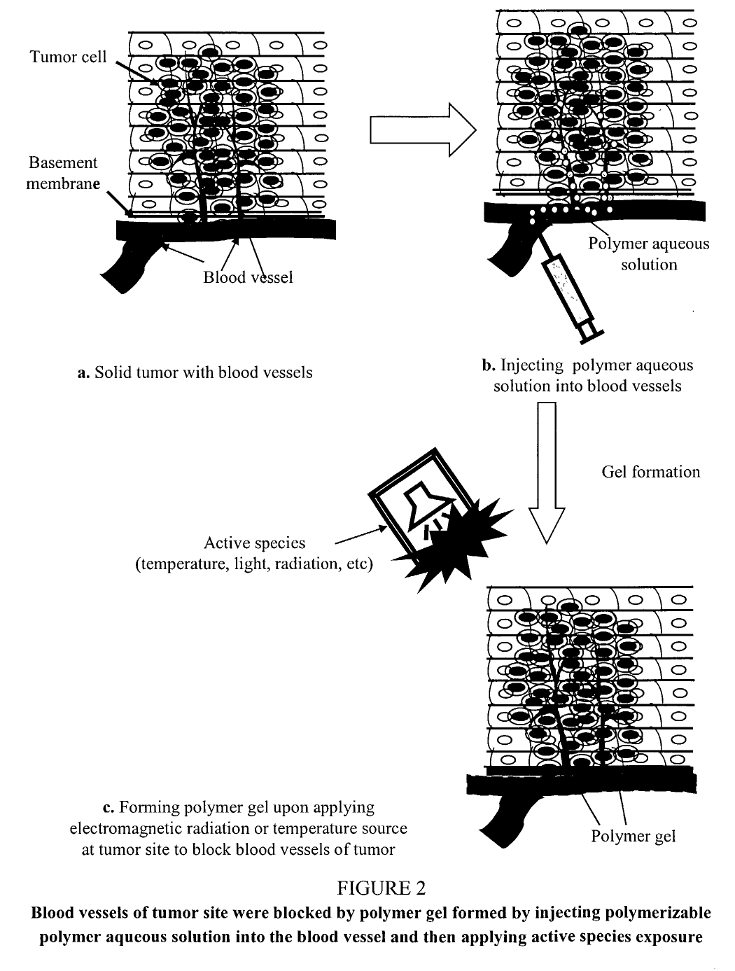

### DETAILED DESCRIPTION OF THE INVENTION

The present invention comprises a method that is to treat cancer by blocking and coating tumor blood vessel through a solid polymer gel formation in tumor blood vessel. A polymer aqueous solution is injected into blood vessel in tumor site and then apply active species source (for example, electromagnetic radiation or temperature) to initiate polymer crosslinking or phase transition. A solid polymer gel is formed in tumor blood vessel by polymer crosslinking or phase transition. The polymer aqueous solution in other place without active species exposure will still be liquid and degraded by hydrolysis or enzyme. The polymer aqueous solution in the blood vessels of tumor undergoes an immediate solidification by exposure to electromagnetic radiation and temperature change at tumor site. The solid polymer gels completely fill the whole blood vessels of tumor and cut off the supply of nutrient and oxygen from blood and migration of tumor cells into blood circulation. The tumor growth will be controlled and finally tumor cells starve and perish because of no nutrient and oxygen supply. The solid polymer gel in blood vessels of tumor is also able to prevent metastasis which causes second cancer, because the gel block tumor cells to shed into blood circulation. The present invention also comprises a method to locally deliver anti-cancer drug into the tumor site by gel formation of polymer aqueous solution with anti-cancer drug in the blood vessels of tumor, following injection of a mixture solution of polymer aqueous solution and anti-cancer drug.

Definitions

A or an, as used herein in the specification, may mean one or more than one. As used herein in the claim(s), when used in conjunction with the word “comprising”, the words “a” or “an” may mean one or more than one.

Blocking, as used herein, refers to the achievement of the filling of whole vessels in tumor site and obstructing blood going through.

Coating, as used herein, refers to the achievement of a layer of solid polymer gel in the wall of blood vessels.

Polymer, as used herein, refers to a macromolecule of many repeating monomer units, making from polymerization of small organic molecules, or some high molecular weight molecules. The polymer may be synthetic or nature macromolecule.

Polymer aqueous solution, as used herein refers to any kind of macromolecules and high molecular weight molecules with or without reaction group (polymerizable, crosslinkable) end groups or with phase transition, which can form gel by crosslinking or phase transition.

Crosslinking, as used herein, refers to covalently conjugation or network formation of inter- or intra-polymer molecule or with tissue wall by using crosslinking agents.

Crosslinking agent, as used herein, refers to a small molecule with bifunctional groups which are able to react with two polymer molecules, such as cocatylast.

Angiogenesis and Tumor Growth

It has long been recognized that most solid tumors contain large numbers of highly permeable blood vessels (Ide, A. G., 1939). These vessels provide nutrients and oxygen of tumor cells for tumor growth and the principal route by which tumor cells exit the primary tumor site and enter the circulation. For many tumors, the vascular density can provide a prognostic indicator of metastatic potential, with the highly vascular primary tumors having a higher incidence of metastasis than poorly vascular tumors. It is now widely recognized that the growth and metastasis of malignant tumors depends blood vessels (Folkman, J., 1997). The vasculature within tumors is distinct (Folkman, J., 1997, Fox, S. B., 1997, Molema, G., 1997), presumably because tumor vessels growing actively match the growth of the tumor. In a startling series of papers in the early 1970s, Folkman (Folkman, J., 1971) laid out the principles that underlie the contemporary era of research in the field of tumor angiogenesis. He hypothesized that the new vessels at the tumor site were not inconsequential but rather that they were absolutely required for expansion of the tumor spheroid beyond a diameter of 1-2 mm, at which point diffusion of nutrients and waste products become rate limiting for continued development of the tumor (Folkman, J., 1971). Folkman postulated that if new blood vessels were indeed essential for tumor growth, then inhibiting angiogenesis should inhibit tumor expansion. If the tumor vessels regressed after treatment, this should cause regression of the tumor mass back to a vascular 1-2 mm spheroid(Folkman, J., 1972)

Tumor angiogenesis is regulated by the production of angiogenic stimulators including members of the fibroblast growth factor (FGF) and vascular endothelial growth factor (VEGF) families (Ferning, D. G., et al., 1994, Dvorak, H. F., et al., 1995, Claffey, K. P., et al., 1996). In addition, tumors may activate angiogenic inhibitors such as angiostatin and endostatin that can modulate angiogenesis both at the primary site and at downstream sites of metastasis (Zetter, B. R., 1998). The potential use of these and other natural and synthetic angiogenic inhibitors as anticancer drugs is still currently under intense investigation. Monoclonal antibodies against tumor antigens have also provided the existing targeting opportunities (Hellstrom, I., et al., 1996; Pastan, I., 1997). However, this approach has met with limited success, because of limited number of known tumor antigens, poorly penetrating into solid tumors and the unstable and mutations of tumor cells (Dvorak, H. F., 1991; Shockley, T. R. et al., 1991; Jain, R. K., 1997; Nowell, P. C., 1976). Moreover, tumor-targeting peptide and polymer materials have been developed to conjugate with anti-cancer drug or anti-angiogenic inhibitor for targeting tumor therapy.

Although these antiangiogenic agents could be useful in causing tumor regression, the most commonly held theories of angiogenesis predict that, in most cases, some tumor may remain after a course of antiangiogenic treatment. That is, the tumor would regress until it reached a small-enough size (<2 mm thick) to be no longer dependent on angiogenesis. The tumor may be expected to stay at this size as long as the treatment is continued. However, if the antiangiogenic treatment were discontinued, the tumor would be expected to regrow.

Also, for all of these methods including other systemic treatment of cytotoxic chemotherapy, a long-term therapy is necessary. And it suffers from the problems of the dose restriction of anticancer agents by their nonselective toxic effects on normal tissue and secondarily acquired drug-resistance. Furthermore, recent all of these methods are not able to eliminate tumor blood vessel and tumor in short time, and finally cause metastasis. In present invention, a rapid and complete blocking or coating of blood vessels of tumor method is provided for cancer therapy.

Polymer Gels for Completely Blocking or Coating Blood Vessels at Tumor Site for Cancer Treatment

I. Polymer Hydrogel

The application of polymeric materials for medical purposes is growing very fast (Jagur-Grodzinski, 1999). Polymers have found applications in such diverse biomedical fields as tissue engineering, implantation of medical devices and artificial organs, prostheses, ophthalmology, dentisty, bone repair, and many other medical fields. Polymer-based delivery systems enable controlled slow release of drugs into the body. They also make possible targeting of drugs into sites of inflammation or tumors.

Polymer hydrogels have been developed for drug delivery system in vivo, because many polymer hydrogel change volume in response to marked changes in environment conditions, such as temperature, solvent, light and pH (Dusek, K., 1993, Okano, T., 1993, Hoffman, A. S., 1995, Tanaka, T., 1981). Moreover, most polymer hydrogels are biocompatible, non- or lower toxic and some of them are biodegradable. Polymer hydrogels have been used to encapsulate cells or drug for delivery into the body.

In present invention, solid polymer gels are disclosed to be used for blocking or coating blood vessels of tumor for cancer therapy. Hydrophilic polymeric aqueous solution with or without crosslinkable groups is injected into the blood vessels and forms gel in blood vessels of tumor when active species (electromagnetic radiation or temperature change) is externally or directly applied to tumor site to induce polymer crosslinking and phase transition of polymer aqueous solution in tumor blood vessels.

II. Composition of Polymer Aqueous Solution for Gel Formation

The polymerizable polymer aqueous solution used in present invention for gel formation in tumor blood vessels may include, but not limited to two categories; photopolymerizable and temperature-responsive gellable polymers. The polymer aqueous solution compositions for gel formation can consist solely of covalently crosslinkable polymers in combination with an effective photoinitiator to allow crosslinking using radiation provided by an external electromagnetic radiation source. Or polymer aqueous solution compositions for gel formation can consist of covalently crosslinkable polymers or temperature responsive gellable polymers to allow gel formation by temperature change provided by temperature source applied at tumor site. The polymer aqueous solution for gel formation can also be mixed or covalently conjugated with anti-cancer drug to locally delivery of anti-cancer drug to tumor, when the polymer aqueous solution forms gel in blood vessels of tumor.

A. Photoinitiation Gellable Polymer Aqueous Solution

The photoploymerization over other cross-linking techniques are spatial and temporal control of the polymerization, fast curing rates at room temperature, and ease of fashioning and flexibility in vivo. The polymer aqueous solution compositions can consist solely of covalently crosslinkable polymer, in combination with an effective but non-toxic photoinitiator to allow crosslinkable using radiation provide by an external source, or blends of covalently and ionically crosslinkable or hydrophilic polymer aqueous solution which form network therein, when exposed to radiation.

In present invention, the photo-crosslinkable polymer aqueous solution comprises a hydrophilic polymer with free radical polymerizable groups, dye and cocatylst.

Exemplary dyes which are mixed with photopolymerizable polymer aqueous solution for gel formation by photoiniatiation include, but not limited to erythrosine, phloxime, rose Bengal, thonine, camphorquinone, ethyl cosin, eosin, methylene blue, riboflavin, 2,2-methyl-2-phenylacetophenone, 2-methoxy-2-phenylacetophenone, 2,2-dimethoxy-2-phenylacetophenone, and other acetophenone derivatives, and camphorquinone.

Suitable cocatalysts include, but not limited to N-methyldiethanolamine, N, N-dimethyl benzylamine, triethanolamine, triethylaimine, dibenzylamine, N-benzylethanolamine, N,N′-methylene-bis-acrylamide, ammonium persulfate and N-isopropyl benzylamine. The preferred cocatalyst is triethanolamine.

Suitable hydrophilic polymers for polymer aqueous solution include, but not limit to these synthetic polymers, such as poly (ethylene glycol), poly(ethylene oxide), partially or fully hydrolyzed poly(vinyl alcohol), poly(vinylpyrrolidone), poly(ethyloxazoline), polyacrylamide and its copolymer with polyacrylate, poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll, RTM, polysucrose, hyaluronic acid, dextran, heparin sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof. These polymers can be modified to contain photo-polymerizable groups. Methods for modifying hydrophilic polymers to include free radical polymerizable groups are well known to those of skill in the art.

Suitable free radical polymerizable groups include ethylenically unsaturated groups (i.e. vinyl groups) such as vinyl ethers, allyl groups, unsaturated monocarbocylic acids, unsaturated dicarboxylic acids, and unsaturated tricarboxylic acids. Unsaturated monocarboxylic acids include acrylic acid, methacrylic acid and crotonic acid, acrylamide. Unsaturated dicarboxylic acids include maleic, fumaric, itaconic, mesaconic or citraconic acid. The photoinitiatable active groups may be located at one or more ends of the hydrophilic polymer, or within block copolymer with one or more hydrophilic polymers forming the individual blocks. The preferred polymerizable groups are acrylates, diacrylates, oligoacrylates, dimethacrylates, oligomethacrylates, and other biologically acceptable photopolymerizable groups. Acrylates are the most preferred active species polymerizable group.

A preferred the polymer aqueous solution containing photo-polymerizable group of the present invention comprise polyethylene glycol (PEG)-based polymers, poly (propylene glycol)(PPG) or poly(tetramethylene glycol)(PTMG)-based polymers and polyanhydrides. Examples of PEG-based photosensitive polymers which polymerize by photosensitive chemicals and or light and or radiation to form a solid gel include, but are not limited to branched PEG-cinnamylidene acetylchloride (b-PEG-CA), polyethylene glycol diacrylate (PEG-DA), PEG-co-polylactic acid and diacrylate (PEG-L-DA). A biocompatible gel can be formed via photopolymerization of PEG-based photosensitive polymer, adhere to the blood vessel wall and block blood vessels.

The synthesis of photosensitive PEG macromer (b-PEG-CA) is well known by the skilled artisan. One published method includes modification of hydroxyl ends of b-PEG with cinnamylidene acetylchloride (Andreopoulos, F. M., et al, 1999). The cinnamylidene acetylchloride can be prepared according to the method of Yamaoka et al (Yamaoka, T., et al, 1977). The b-PEG with cinnamylidene acetate groups (b-PEG-CA) is water-soluble, photosensitive PEG macromers. The b-PEG-CA macromer undergo photo-crosslinking reaction and formed gels upon UV irradiation (>300 nm) in the presence of photoiniatiator, erythrosine B.

PEG-DA is commercial available in Sigma-Aldrich Chemical Company, St. Louis, Mo., USA. PEG-L-DA can be synthesized as described method (Hill-West, J. L., et al, 1994). A highly crosslinked hydrogel of PEG-DA or PEG-L-DA can be formed by the photoinitiation in the presence of eosin and triethanolamine and N-vinylpyrrolidone, as described method (West, J. et al, 1996). And formation of gel of PEG-DA can also be carried out by the UV photo polymerization in the presence of penaerythritol triacrylate and 2,2′-dimethoxy-2-phenyl-acetophenone. Additionally, water soluble polymers which include cinnamoyl groups which may be photochemically crosslinked may be utilized, as described in Matsuda et al., 1992.

The PPG or PTMG photo-polymerizable polymer aqueous solution with acrylate end-capped groups (PPG-A or PTMG-A) can be synthesized as described method (Kim, B. S., et al, 2000). A crosslinking gel of PPG-A or PTMG-A can be formed upon UV-initiated free-radical polymerization.

Photopolymerizable methacrylated anhydride monomers and oligomers can be synthesized from precursor diacid molecules of sebacic acid (SA), 1,3-bis(p-carboxy phenoxy)propane (CPP), and 1,6-bis(p-carboxy phenoxy) hexane (CPH), as described method (Anseth, K., et al, 1999). Upon exposure to UV light, the high concentration of double bonds in these systems and the multifunctional nature of the monomer or oligomer lead to the formation of highly cross-linked polymer networks in a period of seconds to minutes depending on the reaction conditions.

B. Temperature-Responsive Gellable Polymer Aqueous Solution

As used herein, “temperature-responsive gellable polymer aqueous solution” are defined as polymers or high molecular weight molecules which can form solid gel at a temperature of between 0 and 70° C. The solvent herein used to dissolve the polymers include aqueous solution, water-soluble organic solvents, such as dimethylsulfoxide, dimethylformamide, alcohols, acetone, and or mixture.

Suitable synthetic temperature-responsive gellable polymers include synthetic and nature polymers and monomer molecules. Synthetic polymers and monomer molecules include, but not limited to poly (propylene fumarate)(PPF), poly(ethylene glycol)-dimethacrylate (PEG-DMA), N-isopropylacrylamide, acrylic acid and copolymer of N-isopropylacrylamide and acrylic acid. Nature polymers and monomer molecules include, but not limited to polypeptides, polysaccharides or carbohydrates, such as gelatine, collagen, agar, agarose, aliginate, chitosan and their modified derivatives.

A preferred temperature-responsive gellable polymer is PPF. PPF is unsaturated linear polyester with fumarate double bonds that can be crosslinked upon temperature change (Domb, A. J., et al, 1996, Gresser, J. D., et al, 1995). An injectable, in situ polymerizable composite formulations consisting of PPF, PEG-DMA and β-tricalcium phosphate (β-TCP) can form a gel at a temperature just above the body temperature 40° C., as described method (He, S., et al, 2000)

PEG-DMA and β-TCP are commercial available in Sigma-Aldrich Chemical Company (St Louis, Mo.) and Depuy (Warsaw, IN). PPF can be synthesized by a two-step reaction process as a described method (Peter, S. J., et al, 1999). PPF is crosslinked with PEG-DMA as a crosslinking reagent. The maximum crosslinking temperature of PEG-DMA/PPF compositions incorporating β-TCP as well as that of PEG-DMA/PPF networks is not affected by the PEG-DMA/PPF ratio or β-TCP content, and is about 40° C. The crosslinking density of PEG-DMA/PPF networks increased with the PEG-DMA/PPF double-bond ratio resulting in increased mechanical properties of PEG-DMA/PPF networks and crosslinked composites (He, S., et al, 2000)

Exemplary temperature responsive gellable polymers which can be used to form a gel also include, but not limited to modified alginates, gelatine, agar, agarose, chitosan and their derivatives. Naturally occurring alginate may be chemically modified to produce alginate polymer derivatives. Modified alginate may be gellable with other another selected moiety, such as, lactic acid. Modified hyaluronic acid derivatives are particularly useful. Modified hyaluronic acid may be designed and synthesized which are esterified with a relatively hydrophobic group such as propionic acid or benzylic acid to render the polymer more hydrophobic and gel forming. Hyaluronic acid and hyaluronic derivatives are commercial available. Preferred temperature gellable nature polymeric precursors are gelatine, agar and agarose. These nature polymers are dissolved in water when they are heated and form solid gel when they are cooled.

Another preferred nature polymer for gellable polymer aqueous solution is chitosan. Chitosan/polyol salt combinations formulation can be held liquid below room temperature in a physiological pH and form monolithic gels if heated at body temperature, as a described method (Chenite, A., et al, 2000). Therefore, chitosan/polyol salt formulation may be useful to form solid gel in the blood vessels of tumor to block whole tumor blood vessels by modifying the formulation to adjust the gelation temperature to above body temperature.

C. Electronic Field-Responsive Gellable Polymer Aqueous Solution

Polymer hydrogel in polymer aqueous solution may also be formed by external electric current stimuli: Electrically stimuli gellable polymer include, but not limited to poly (ethyloxazoline)(PEOx) and either poly (methacrylic acid)(PMMA) or poly (acrylic acid).

2. Injection of Gellable Polymer Aqueous Solution into Blood Vessels at Tumor Site

In the preferred embodiment, the gellable polymer aqueous solution with other selected reagents (crosslinking agents, dye, cocatylst, anti-cancer drug) may be injected directly into blood vessels of tumor, and form solid gel by crosslinking and gel formation of the polymer.

The tumor site, or sites, where mixed solution is to be injected is determined based on the individual need, such as tumor position, size of tumor blood vessel and organ. An example of injection is directly injecting solution via syringe and needle into the artery closed to the tumor site and solution infuses into tumor blood vessels. Or injection may be directly at tumor site to penetrate into blood vessels of tumor.

A preferred injection system is infusion injection system. Mixed solution can be regional infused through artery into blood vessel at tumor site with the infusion system as described by Conn, H, et al (Conn, H., et al, 1978). The infusion system may be thus arranged to deliver higher concentration of polymer aqueous solution at the tumor site by positioning a silicone catheter in the common artery.

III. Application of Active Species Generators

In the embodiment, an electromagnetic radiation is disclosed as a radiation source to initiate photo-polymerization of polymeric precursor. The electromagnetic radiation source can be applied to tumor tissue, or as an external source to penetrate tissue to cause photo-polymerization.

“Electromagnetic radiation” herein refers to energy waves of the electromagnetic spectrum including, but not limited to, x-ray, y-ray, ultraviolet, visible, infrared, far infrared, microwave, laser and radiation frequency.

In the embodiment using photo-polymerizable polymer aqueous solution, the electromagnetic radiation source is applied externally or directly to the tumor tissue where the polymer aqueous solution has been injected or infused. Polymeric precursors with active molecules can be crosslinked by active species generation, photo-activation.

The depth of penetration can be controlled by the wavelength of the light utilized to cause the photopolymerization, such as visible light penetrates deeper through tissue than UV light. Also, other electromagnetic radiation sources which can produce light to cause photopolymerization may be used.

In the embodiment using temperature-responsive gellable polymer aqueous solution, the temperature source is applied directly to the tumor tissue where the polymer aqueous solution has been injected or infused. Then, the polymer aqueous solution form solid gels by cooling temperature down or crosslinks to form gel by heating. The temperature source may be heater, cooler, liquid gas, solid gas and ultrasound.

IV. Local Delivery of Anti-Cancer Drug with Polymer Gel

Polymer aqueous solution may be mixed with anti-cancer drug and then injected into the blood vessels of tumor site. After applying electromagnetic radiation or temperature at tumor site, the polymer aqueous solution forms a solid gel and anti-cancer drug is encapsulated inside the gel in blood vessels of tumor. The anti-cancer drug may also be covalently conjugated with the polymer in polymer aqueous solution. After applying electromagnetic radiation or temperature at tumor site, the polymer aqueous solution forms a solid gel and anti-cancer drug is encapsulated inside the gel in blood vessels of tumor. The anti-cancer drugs locally release into tumor site. Herein, anti-drugs include chemotherapy drug, radiation drug, anti-angiogenic drug. Exemplary of chemotherapy drugs include, but not limited to these, doxorubicin, cisplatin, etoposide, vinblastine, vincristine, estrumustine, suramin, staurosporine and paclitaxel, etc. Exemplary of anti-angiogenic drug include, but not limited to these, angiogenstatin, estastatin and other anti-angiogenic protein, polypeptide.

V. Coating of Blood Vessel of Tumor by Polymer Gel

The inside wall of tumor blood vessel can also be just coated by solid polymer gel to block the penetration of nutrient from blood to the tumor cell and migration of tumor cell into blood circulation. A polymer gel coating barrier in the walls of blood vessels of tumor can be created by using a tissue-adsorbing photoinitiator, eosin Y. Eosin Y can stick to blood vessel wall. After photoinitiation, Eosin Y can crossslink with polymer aqueous solution by photopolymerizable group. Examples of the method are that a polymer aqueous solution of polyethylene glycol diacrylate (PEG-DA; nondegradable) or PEG-co-polylactic acid diacrylate (PEG-L-DA; degradable) with cocatalysts triethanolamine and N-vinylpyrrolidone and eosin Y is injected into blood vessel and than illuminated with a xenon arc lamp at tumor tissue, as described method (West, J. et al, 1996). A solid polymer gel layer will be tightly coated on the blood vessel walls of tumor to block the nutrients supply of tumor cells and tumor cells to shed into blood circulation.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Andreopoulos, F. M., Roberts, M. J., Bentley, M. D., Harris, J. M., Beckman, E. J., and Russell, A. J. (1999), Photoimmobilization of organophosphorus hydrolase within a PEG-based hydrogel. Biotechnol Bioeng., 65, 579-588.

Anseth, K. S., Shastri, V. R., and Langer, R. (1999), Photopolymerizable degradable polyanhydrides with osteocompatibility, Nature Biotechnology, 17, 156-159.

Arap, W., Pasqualini, R., & Ruoslahti, E. (1998), Cancer treatment by targeted drug delivery to tumor vasculature in a mouse model, Science, 279, 377-380.

Arap, W., Pasqualini, R., Ruoslahti, E. (1998), Chemotherapy targeted to tumor vasculature, Current Opinion in Oncology, 10, 560-565.

Belotti, D., Vergani, V., Drudis, T., Borsotti, P., Pitelli, M. R., Viale, G., Giavazzi, R., and Taraboletti, G.(1996), The microtubule-affecting drug Paclitaxel has antiangiogenic activity. Clin. Cancer Res., 2, 1843-1849.

Bicknell, R.(1997), in Tumor angiogenesis. Eds. Bicknell, R., Lewis, C. E. & Ferrara, N., Oxford University Press, Oxford, 19-28.

Brooks, P. C., Clark, R. A., Cheresh, D. A. (1994), Requirement of vascular integrin alpha v beta 3 for angiogenesis, Science, 264, 569-571.

Brooks, P. C., Montgomery, A. M., Rosenfeld, M., et al.(1994), Integrin alpha v beta 3 antagonists promote tumor regression by inducing apoptosis of angiogenic blood vessels., Cell, 79, 1157-1164.

Chenite, A., Chaput, C., Wang, D., Combes, C., Buschmann, M. D., Hoemann, C. D., Leroux, J. C., Atkinson, B. L., Binette, F. and Selmani, A.(2000), Novel injectable neutral solutions of chitosan form biodegradable gels in situ. Biomaterials, 21, 2155-2161.

Claffey, K. P., Robinson, G. S.(1996), Regulation of VEGF/VPF expression in tumor cells: consequences for tumor growth and metastasis., Cancer Metastasis Rev., 15, 165-176.

Conn, H. & Langer, R. (1978), Continuous long-term intra-arterial infusion in the unrestrained rabbit, Lab. Anim. Sci., 28, 598-602.

Domb, A. J., Manor, N., Elmalak, O.(1996), Biodegradable bond cement compositions based on acrylate and epoxide terminated poly(propylene fumarate) oligomers and calcium salt compositions, Biomaterials, 17, 411-417.

Dusek, K.(ed) (1993), Responsive Gels: Volume Phase Transitions, Advances in Polymer Science, 109, and 110 (Springer, Berlin)

Dvorak, H. F., Brown, L. F., Detmar, M., Dvorak, A. M.(1995), Vascular permeability factor/vascular endothelial growth factor, microvascular hyperpermeability, and angiogenesis., Am. J Pathol., 146, 1029-1039.

Dvorak, H. F., Nagy, J. A., Dvorak, A. M.(1991), Structure of solid tumors and their vasculature: implication for therapy with monoclonal antibodies. Cancer Cells, 3, 77-85.

Elisseeff, J., McIntosh, W., Anseth, K., Riley, S., Ragan, P., and Langer, R.(2000), Photoencapsulation of chondrocytes in poly(ethylene oxide)-based semi-interpenetrating networks, J. Biomed. Mater Res, 51, 164-171.

Ferning, D. G., Gallaher, J. T.(1994), Fibroblast growth factors and their receptors: an information network controlling tissue growth, morphogenesis and repair. Prog. Growth Factor Res., 5, 353-377.

Figg, W. D., Pluda, J. M., Lush, R. M., et al.(1997), The Pharmacokinetics of TNP-470, a new angiogenesis inhibitor., Pharmacotherapy, 17, 91-97.

Folkman, J.(1971), Tumor angiogenesis: therapeutic implications, N. Engl. J. Med., 285, 1182-1186.

Folkman, J.(1974), in Advances in Cancer Research, eds. Klein, G. & Weinhous, S. (Academic, New York), vol. 19, pp. 331-358.

Folkman, J.(1972), Anti-angiogenesis: new concept for therapy of solid tumors, Ann. Surg., 175, 409-416.

Folkman, J.(1997), in Cancer: Principles and Practice of Oncology., eds. DeVita, V. T., Hellman, S. & Rosenberg, S. A., Lippincott-Raven, New York, 3075-3087.

Folman, J. and Hochberg, M.(1973), Self-regulation of growth in three dimensions, J. Exp. Med., 138, 745-753.

Folkman, J., Langer, R., Linhardt, R. J., Haudenschild, C., and Taylor, S.(1983), Angiogenesis inhibition and tumor regression caused by heparin or a heparin fragment in the presence of cortisone, Science, 221, 719-725.

Fox, S. B., Harris, A. L.(1997), Markers of tumor angiogenesis: clinical applications in prognosis and anti-angiogenic therapy., Invest. New Drugs, 15, 15-28.

Fujioka, K., Maeda, M., Hojo, T., and Sano, A.(1998), Protein release from collagen matrices, Advanced Drug Delivery Reviews, 31, 247-266.

Gagliardi, A. R., Henning, B., Collins, D. C.(1996), Antiestrogens inhibit endothelial cell growth stimulated by angiogenic growth factors., Anticancer Res., 16, 1101-1106.

Gresser, J. D., Hsu, S. H., Nagaoka, H., Lyons, C. M., Nieratko, D. P., Wise, D. L., Barabino, G. A., Trantolo, D. J.(1995), Analysis of a vinyl pyrrolidine/poly(propylene fumarate) restorable bone cement. J. Biomed Mater Res, 29, 1241-1247.

He, S., Yaszemski, M. J., Yasko, A. W., Engel, P. S., and Mikos, A. G. (2000), Injectable biodegradable polymer composites based on poly(propylene fumarate) crosslinked with poly(ethylene glycol)-dimethacrylate, Biomaterial, 21, 2389-2394.

Hellstrom, I., Trail, P., Siegall, C., Firestone, R., Hellstrom, K. E.(1996), Immunoconjugates and immunotoxins for therapy of solid tumors, Cancer Chemother Pharmacol, 38, 35-36.

Hember, M. W. N., Richardson, R. K., and Morris, E. R.(1994), Native ordered structure of welan polysaccharide: conformational transitions and gel formation in aqueous dimethyl sulphoxide, Carbohydrate Research, 252, 209-221.

Hikmet, R. A. M., and Kemperman, H.(1998), Electrically switchable mirrors and optical components made from liquid-crystal gels, Nature, 392, 476-482.

Hill-West, J. L., Chowdhury, S. M., Slepian, M. L. & Hubbell, J. A.(1994), Inhibition of thrombosis and intimal thickening by in situ photopolymerization of thin hydrogel barriers, Proc. Natl. Acad. Sci. USA, 91, 5967-5971.

Hoffman, A. S. (1995), “Intelligent” polymers in medicine and biotechnology, Artificial Organs, 19, 458-467.

Holtz, J. H., and Asher, S.(1997), Polymerized colloidal crystal hydrogel films as intelligent chemical sensing materials, Nature, 389, 829-832.

Ide, A. G., Baker, N. H., Warren, S. L.(1993), Vascularization of the Brown-Pearce rebbit epithelioma transplant as seen in the transparent ear chamber., Am. J. Roentgenol, 42, 891-899.

Jagur-Grodzinski, J.(1999), Biomedical application of functional polymers, Reactive & Functional Polymers, 39, 99-138.

Jain, R. K.(1997), Delivery of molecular and cellular medicine to solid tumors, Microcirculation, 4, 3-23.

Juodkazis, S., Mukai, N., Wakaki, R., Yamaguchi, A., Matsuo, S., and Misawa, H. (2000), Reversible phase transitions in polymer gels induced by radiation forces, Nature, 408, 178-181.

Kim, B. S., Hrkach, J. S., and Langer, R.(2000), Biodegradable photo-crosslinked poly(ether-ester) networks for lubricious coatings. Biomaterials, 21, 259-265.

Kohn, E. C., Reed, E., Sarosy, G. et al.(1996), Clinical investigation of a cytostatic calcium influx inhibitor in patients with refractory cancers., Cancer Res., 56, 569-573.

Koivunen, E., Arap, W., Valtanen, H., Rainisalo, A., Medina, O. P., Heikkila, P., Kantor, C., Gahmberg, C. G., Salo, T., Konttinen, Y. T., Sorsa, T., Ruoslahti, E., and Pasqualini, R. (1999), Tumor targeting with a selective gelatinase inhibitor, Nature Biotechnology, 17, 768-774.

Kwon, I. C., Bae, Y. H., and Kim, S. W. (1991), Electrically erodible polymer gel for controlled release of drugs, Nature, 354, 291-293.

Langer, R., Brem, H., Falterman, K., Klein, M. and Folkman, J. (1976), Isolation of a cartilage factor that inhibitors tumor neovascularization. Science, 193, 70-72.

Langer, R., Conn, H., Vacanti, J., Haudenschild, C., and Folkam, J. (1980), Control of tumor growth in animals by infusion of an angiogenesis inhibitor. Proc. Natl. Acad. Sci. USA, 77, 4331-4335.

Langer R. and Folkman, J. (1976), Polymers for the sustained release of proteins and other macromolecules, Nature, 263, 797-800.

Langer, R., and Murray, J.(1983), Angiogenesis inhibitors and their delivery system, Appl. Biochem. Biotechn., 8, 9-24.

Lele, B. S., and Hoffman, A. S.(2000), Mucoadhesive drug carriers based on complexes of poly(acrylic acid) and PEGylated drugs having hydrolysable PEG-anhydride-drug linkages, J. Controlled Release, 69, 237-248.

Matsuda et al.(1992), ASAID Trans., 38, 154-157.

Mellott, M. B., Searcy, K., and Pishko, M. V. (2001), Release of protein from highly cross-linked hydrogels of poly(ethylene glycol) diacrylate fabricated by UV polymerization, Biomaterials, 22, 929-941.

Molema, G., de Leij, L. F., & Meijer, D. K. (1997), Tumor vascular endothelium: barrier or target in tumor directed drug delivery and immunotherapy., Pharm. Res., 14, 2-10.

Moses, M. A., and Langer, R. (1991), Inhibitors of angiogenesis, Biotechnology, 9, 630-634.

Nowell, P. C. (1976), The clonal evolution of tumor cell populations, Science, 194, 23-28.

Okano, T.(1993), Molecular design of temperature-responsive polymers as intelligent materials. Adv. Polym. Sci., 110, 179-197.

O'Reilly, M. S., Boehm, T., Shing, Y., et al. (1997), Endostatin: an endogenous inhibitor of angiogenesis and tumor growth. Cell, 88, 1-20.

O'Reilly, M. S., Homgren, L., Shing, Y., et al. (1994), Angiostatin: a novel angiogenesis inhibitor that mediates the suppression of metastases by a Lewis lung carcinoma. Cell, 79, 315-328.

Pasqualini, R., Koivunen, E., Ruoslahti, E.(1997), Alpha-v integrins as receptors for tumor targeting by circulating ligands, Nat. Biotechnol., 15, 542-546.

Pasqualini, R., and Ruoslahti, E. (1996), Organ targeting in vivo using phage display peptide libraries, Nature, 380, 364-366.

Pastan, I.(1997), Targeted therapy of cancer with recombinant immunotoxins, Biochim Biophys Acta, 1333, 1-6.

Peter S. J., Kim P., Yasko A. W., Yaszemski M. J., Mikos A. G.(1999), Cross-linking characteristics of an injectable poly(propylene fumarate)/b-tricalcium phosphate past and mechanical properties of the cross-linked composite for use as a biodegradable bone cement, J. Biomed Mater Res., 44, 314-321.

Petka, W. A., Harden, J. L., McGrath, K. P., Wirtz, D., and Tirrell, D. A. (1998), Reversible hydrogels from self-assembling artificial protins, Science, 281, 389-392.

Pollard, T. D. (1976), The role of actin in the temperature-dependent gelation and intraction of extracts of acanthamoeba, J. Cell Biol., 68, 579-601.

Risau, W. (1997), Mechanisms of angiogenesis. Nature, 386, 671-674.

Shockley, T. R., Lin, K., Nagy, J. A, Tompkins, R. G., Dvorak, H. F., Yamush, M. L.(1991), Penetration of tumor tissue by antibodies and other immunoproteins., Ann N Y Acad Sci., 618, 367-382.

Steiner, R. (1992), Angiostatic activity of anticancer agents in the chick embryo chorioallantoic membrane (CHE-CAM) assay, In Angiogenesis: Key Principles-Science-technology-Medicine, ed., Steiner, R., Weiss, P., Langer, R., 2, 449-454.

Teicher, B. A., Holden, S. A., Ara, G., et al. (1994), Potentiation of cytotoxic cancer therapies by TNP-470 alone and with other anti-angiogenic agents, Int. J. Canecr, 57, 920-925.

Teicher, B. A., Sotomayor, E. A., Huang, Z. D. (1992), Antiangiogenic agents potentiate cytotoxic cancer therapies against primary and metastatic disease. Cancer Res., 52, 6702-6704.

Venter, J. C. (1982), Immobilization and insolubilized drugs, hormones, and neurotransmitters: properties, Mechanisms of action and applications, Pharmacological Review, 34, 153-187.

West, J. L., and Hubbell, J. A. (1996), Separation of the arterial wall from blood contact using hydrogel barriers reduces intimal thickening after balloon injury in the rat: the roles of medical and luminal factors in arterial healing. Proc. Natl. Acad. Sci. USA, 93, 13188-13193.

Westhaus, E., and Messersmith, P. B. (2001), Triggered release of calcium from lipid vesicles: a bioinspired strategy for rapid gelation of polysaccharide and protein hydrogels, Biomaterials, 22, 453-462.

Yamaoka, T., Ueno, K., Tsunoda, T., Torige, K. (1977), A study on phenoxy-resin esters of cinnamylideneacetic acid and its derivatives, Polymer, 18, 81-86.

Yang, J-M., Lu, C.-S., Hsu, Y-G, and Shih, C-H. (1997), Mechanical of acrylic bone cement containing PMMA-SiO₂ hybrid sol-gel material, J. Biomed Mater Res., 38, 143-154.

Zetter, B. R.(1998), Angiogenesis and tumor metastasis. Annu. Rev. Med., 49, 407-424.

One of skill in the art readily appreciates that the present invention is well adapted to carry out the objectives and obtain the ends and advantages mentioned as well as those inherent therein. Methods, procedures and techniques described herein are presently representative of the preferred embodiments and are intended to be exemplary and are not intended as limitations of the scope. Changes herein and other uses will occur to those skilled in the art which are encompassed within the spirit of the invention or defined by the scope of the pending claims.

We claim:

1. A method of treating a solid tumor comprising the steps of:
a. providing a polymer aqueous solution comprising one or more crosslinkable polymers and dye and cocatylst;
b. injecting the said polymer aqueous solution into blood vessels at tumor site of cancer patient or animal; and
c. applying electromagnetic radiation source or heating source externally or directly to the tumor site to generate free radicals which induce polymer crosslinking and gel formation in blood vessels of tumor to block or coat the blood vessels of tumor, and then to cut off nutrients a supply of tumor, and finally starve and perish tumor for cancer therapy.

2. A method of treating a solid tumor comprising the steps of:
a. providing a polymer aqueous solution comprising one or more temperature responsive gellable polymer;
b. injecting the said polymer aqueous solution into blood vessels at tumor site of cancer patient or animal; and
c. applying temperature source externally or directly to the tumor tissue to cause temperature change (increase or decrease temperature) which induce phsae transition of polymer aqueous solution and solid gel formation in blood vessels of tumor to block or coat the blood vessels of tumor, and then to cut off nutrients supply of tumor, and finally starve and perish tumor for cancer therapy.

3. The method of claim 1 wherein the electromagnetic radiation source is, but not limited to x-rays, ultrasound, infrared radiation, far infrared radiation, ultraviolet radiation, long-wavelength ultraviolet radiation, visible light, laser beam and γ-ray radiation.

4. The method of claim 1 wherein the heating source is, but not limited to heater, and ultrasound, etc.

5. The method of claim 1 wherein the gellable polymer aqueous solution further comprises a photoinitiator.

6. The method of claim 1 wherein the gellable polymer aqueous solution further comprises a cocataylst.

7. The method of claim 1 wherein the photoinitiator is selected from the group consisting of erythrosine, phloxime, rose Bengal, thonine, camphorquinone, ethyl eosin, eosin, methylene blue, riboflavin, 2,2-methyl-2-phenylacetophenone, 2-methoxy-2-phenylacetophenone, 2,2-dimethoxy-2-phenylacetophenone, and other acetophenone derivatives.

8. The method of claim 1 wherein the cocatalyst is, but not limited to N-methyldiethanolamine, N, N-dimethyl benzylaime, triethanolamine, triethylaimine, dibenzylamine, N-benzylethanolamine, and N-isopropyl benzylamine.

9. The method of claim 1 wherein the gellable polymer by radiation is these synthetic or nature polymers or high molecular weight molecules with photopolymerizable groups. Synthetic polymer or high molecular weigh molecules can be poly (ethylene glycol), poly(ethylene oxide), partially or fully hydrolyzed poly(vinyl alcohol), poly(vinylpyrrolidone), poly(ethyloxazoline), polyacrylamide and its copolymer with polyacrylate, poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose. Natural polymers and high molecular weigh molecules can be polypeptides, polysaccharides or carbohydrates such as Ficoll, RTM, polysucrose, hyaluronic acid, dextran, heparin sulfate, chondroitin sulfate, heparin, or alginate, and proteins such as gelatin, collagen, albumin, or ovalbumin or copolymers or blends thereof. The photopolymerizable groups include ethylenically unsaturated groups (i.e. vinyl groups) such as vinyl ethers, ally groups, unsaturated monocarbocylic acids. Unsaturated dicarboxylic acids, and unsaturated tricarboxylic acids. Unsaturated monocarboxylic acids include acrylic acid, methacrylic acid and crotonic acid, acrylamide. unsaturated dicarboxylic acids include maleic, fumaric, itaconic, mesaconic or citraconic acid.

10. The method of claim 1 wherein the gellable polymer by heat include, but not limited to these synthetic or nature polymers or high molecular weigh molecules, such as polypeptide, polysaccharide and carbohydrate, and other synthetic polymers or high molecular weigh molecules, such as poly (propylene fumarate)(PPF), poly (ethylene glycol)-dimethacrylate (PEG-DMA), β-tricalcium phosphate (β-TCP), copolymer of N-isopropylacrylamide, acrylic acid, alginate, chitosan and their modified derivatives, modified hyaluronic acid and Chitosan/polyol salt combinations formulation

11. A preferred photopolymerizable polymer in polymer aqueous solution of claim 1 include, but not limited to photosensitive PEG polymer (branched PEG-cinnamylidene acetylchloride, b-PEG-CA), polyethylene glycol diacrylate (PEG-DA), or PEG-co-polylactic acid diacrylate (PEG-L-DA; degradable).

12. The method of claim 1 wherein the injection is, but not limited to artery infused injection or directly blood vessel of tumor injection or tumor site injection.

13. The method of claim 2 wherein the temperature source is, but not limited to heater, cooler, liquid gas, solid gas and ultrasound,

14. The method of claim 2 wherein the gellable polymer in polymer aqueous solution include, but not limited to these nature polymers(polypeptide, polysaccharide and carbohydrate), such as gelatine, agar and agarose and other synthetic polymers

15. The method of claim 2 wherein the injection is, but not limited to artery infused injection or directly blood vessel of tumor injection or tumor tissue injection.

16. A method of locally delivery of anti-cancer drug to tumor site using polymer gel comprising the steps of:
a. providing a gellable polymer aqueous solution in which anti-cancer drugs are also mixed or conjugated with gellable polymeric precursor.
b. injecting the said solution into the blood vessels.
c. applying electromagnetic radiation or temperature source external or direct to the tumor tissue and inducing solid gel formation of polymer aqueous solution in blood vessels of tumor and anti-cancer drug locally releasing from the polymer gel to kill tumor cells.
wherein anti-cancer drug can be physically encapsulated in the polymer gel or covalently conjugated with polymer. After polymer gel formation in blood vessels of tumor upon applying active species (electromagnetic radiation or temperature) at tumor tissue, physically encapsulated anti-cancer drug can locally release to tumor by diffusion, or covalently conjugated anti-cancer drug can locally release to tumor by cleaving the covalent bond between drug and polymer upon on electromagnetic radiation or temperature.

17. The method of claim 16 wherein the anti-cancer drug is selected the group consisting chemotherapy drug, radiation drug or anti-angiogenic drug. Wherein said chemotherapy drug is, but not limited to doxorubicin, cisplatin, etopside, vinblastine and toxiten. Wherein said radiation drug include, but not limited to radiation element and bead. Wherein said anti-angiogenic drug include, but not limited to angiogenstatin, estastatin and other anti-angiogenic protein, polypeptide.

18. The method of claim 16 wherein the electromagnetic radiation source is same as claim 3. Temperature source is same as claim 4 and 13.

19. The method of claim 16 where polymer aqueous solution composition is same as claim 9, 10, 11 and 14.
